(19) United States
(12) Patent Application Publication
(10) Pub. No.: US 2015/0008314 A1
(43) Pub. Date: Jan. 8, 2015

(54) DIAGNOSTIC AND PROGNOSTIC BIOMARKERS FOR CANCER

(71) Applicant: THE CLEVELAND CLINIC FOUNDATION, Cleveland, OH (US)

(72) Inventors: Daniel I. Sessler, Hunting Valley, OH (US); Jinbo Liu, Beachwood, OH (US)

(73) Assignee: THE CLEVELAND CLINIC FOUNDATION, Cleveland, OH (US)

(21) Appl. No.: 14/373,384

(22) PCT Filed: Jan. 25, 2013
(86) PCT No.: PCT/US2013/023145

§ 371 Date: Jul. 21, 2014

Related U.S. Application Data
(60) Provisional application No. 61/590,961, filed on Jan. 26, 2012.

(51) Int. Cl. G01N 33/574; H01J 49/00; H01J 49/16; C12Q 1/44

(52) U.S. Cl. CPC G01N 33/57488; C12Q 1/44; H01J 49/0027; H01J 49/165; G01N 2800/54; G01N 2800/7028
USPC 250/282; 436/501; 435/19

(57) ABSTRACT

A method for detecting cancer from a biological sample previously withdrawn from the subject, in a subject includes determining in vitro the level of at least one biomarker in the biological sample. The at least one biomarker includes a phospholipid or a free fatty acid. A level of the at least one biomarker that is 2-fold greater than the level of at least one biomarker in a control is indicative of cancer in the subject.

### RELATED APPLICATION

This application claims the benefit of U.S. Provisional Patent Application Ser. No. 61/590,961, filed Jan. 26, 2012, the entirety of which is hereby incorporated by reference for all purposes.

### TECHNICAL FIELD

The present disclosure relates generally to biomarkers, methods, and kits for detecting diseases and physiological conditions. More particularly, the present disclosure relates to biomarkers of cancer, methods, and kits for using these biomarkers for diagnostic and prognostic purposes.

### BACKGROUND

Cancer is the second leading cause of death in the United States, and the leading cause of death worldwide. Surgery is the primary curative treatment for most cancers, but it is most successful at early stages. By the time cancer becomes symptomatic, the best chance for surgical cure is usually well past. However, cancer diagnosis at early stages remains challenging.

There are currently few reliable simple screening tests for early cancer detection. For example, bronchogenic carcinoma is the most common cause of lung cancer. It is difficult to diagnose from routine chest X-rays because it typically develops in the hilum behind the heart, and annual chest X-rays do not reduce lung cancer mortality. The only current reliable method of screening for bronchogenic cancer is computerized tomography (CT). However, spiral CT—the best method for detecting lung cancer—is expensive and exposes patients to considerable radiation. Furthermore, 94.5% of nodules identified by CT are benign. CT is thus sub-optimal as a routine screening measure, even in high-risk populations.

Additionally, there is currently no simple and reliable screening test for colon cancer and renal cancer in current clinical practice. In fact, colonoscopy (which is an invasive and expensive procedure) remains the only reliable test for colon cancer.

### SUMMARY

One aspect of the present disclosure includes a method for detecting early stage cancer in a subject from a biological sample previously withdrawn from the subject. One step of method includes determining in vitro the level of at least one biomarker in the biological sample. The at least one biomarker includes a phospholipid or a free fatty acid. A level of the at least one biomarker that is at least about 2-fold greater than the level of at least one biomarker in a control is indicative of early stage cancer in the subject.

Another aspect of the present disclosure can include a method for predicting the prognosis of a subject with cancer following a medical intervention to treat the cancer from a biological sample previously withdrawn from the subject. One step of the method can include determining in vitro the level of at least one biomarker in the biological sample. The at least one biomarker can include a phospholipid or a free fatty acid. A decreased level of the at least one biomarker as compared to a pre-treatment level of at least one biomarker is indicative of a favorable prognosis.

Another aspect of the present disclosure can include a method for detecting cancer recurrence in a subject following a medical intervention to treat the cancer from a biological sample previously withdrawn from the subject. One step of the method can include determining in vitro the level of at least one biomarker in the biological sample over a period of time. The at least one biomarker can include a phospholipid or a free fatty acid. A level of the at least one biomarker that is at least about 2-fold higher than a pre-treatment level of at least one biomarker over the period of time is indicative of cancer recurrence in the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a process flow diagram illustrating a method for detecting early stage cancer in a subject according to one aspect of the present disclosure;

FIG. 2 is a process flow diagram illustrating a method for predicting the prognosis of a subject with cancer following a medical intervention to treat the cancer according to another aspect of the present disclosure;

FIG. 3 is a process flow diagram illustrating a method for detecting cancer recurrence in a subject following a medical intervention to treat the cancer according to another aspect of the present disclosure;

FIG. 4 is a series of boxplots showing a comparison between median values of free fatty acids (FFA) and their metabolites at baseline (log scale) by cancer status in lung cancer patients;

FIG. 5 is a series of Receiver Operating Characteristic (ROC) curves for each of the FFAs and their metabolites in FIG. 4 at baseline (presented as area-under-the-curve (SE));

FIG. 6 is a series of boxplots comparing the levels of the FFAs and their metaoblites in FIG. 4 following lung tumor resection;

FIG. 7 is a series of histograms comparing the level of lysophosphatidylcholine (LPC-C16) in lung cancer and non-cancer patient controls (upper histogram), as well as the level of LPC-C16 following tumor resection (lower histograms);

FIG. 8 is a series of boxplots comparing the levels of FFAs and their metabolites at baseline (log scale) by lung adenocarcinoma cancer status (all p<0.001);

FIG. 9 is an ROC curve for each of the FFAs and their metabolites shown in FIG. 8 (candidate predictors needed to have an area under the curve (AUC) >0.50 and both sensitivity and specificity >0.70 at a best cutpoint that maximized both parameters);

FIG. 10 is a series of boxplots comparing the levels of FFAs and their metabolites in patients with early stage prostate cancer at baseline (log scale) by cancer status (all p<0.001);

FIG. 11 shows a series of ROC curves for patients with early stage prostate cancer with AUC and standard error in parentheses (displayed are the four biomarkers that had AUC significantly >0.50 and both sensitivity and specificity estimated as >0.70);

FIGS. 12A-B are a series of histograms showing FFAs and their metabolites between colon cancer and non-colon cancer patients before (FIG. 12A) and 24-hours after (FIG. 12B) surgery;

FIG. 13 shows a series of boxplots comparing the levels of FFAs and their metabolites in patients with early stage colon cancer at baseline (log scale) by cancer status (all p<0.001);

FIG. 14 is a series of histograms comparing the levels of FFAs and their metabolites in patients with early stage renal cancer at baseline (log scale) by cancer status (all p<0.001);

FIG. 15 shows a series of boxplots comparing the levels of FFAs and their metabolites in patients with early stage renal cancer at baseline (log scale) by cancer status (all p<0.001); and

FIG. 16 is a series of boxplots showing oxidized phospholipid hydroxyoctadecadienoyl phosphatidylcholine (HODS-PC) at baseline (log scale) by all four types of cancer status (all p<0.001).

### DETAILED DESCRIPTION

The present disclosure relates generally to biomarkers, methods, and kits for detecting diseases and physiological conditions. More particularly, the present disclosure relates to biomarkers of cancer, methods, and kits for using these biomarkers for diagnostic and prognostic purposes. The present disclosure is based, at least in part, on the discovery that: (1) specific biomarkers had significantly elevated levels in early stage cancer patients when compared to non-cancer controls; and (2) the biomarker levels were substantially reduced in just 24 hours after tumor resection. Based on this discovery, the present disclosure provides non-invasive and cost effective methods and kits for routine cancer screening to detect early stage malignant tumors, perioperative testing for surgical outcomes, and follow-on testing for prognosis and/or recurrence after a medical intervention (e.g., tumor resection).

### Methods

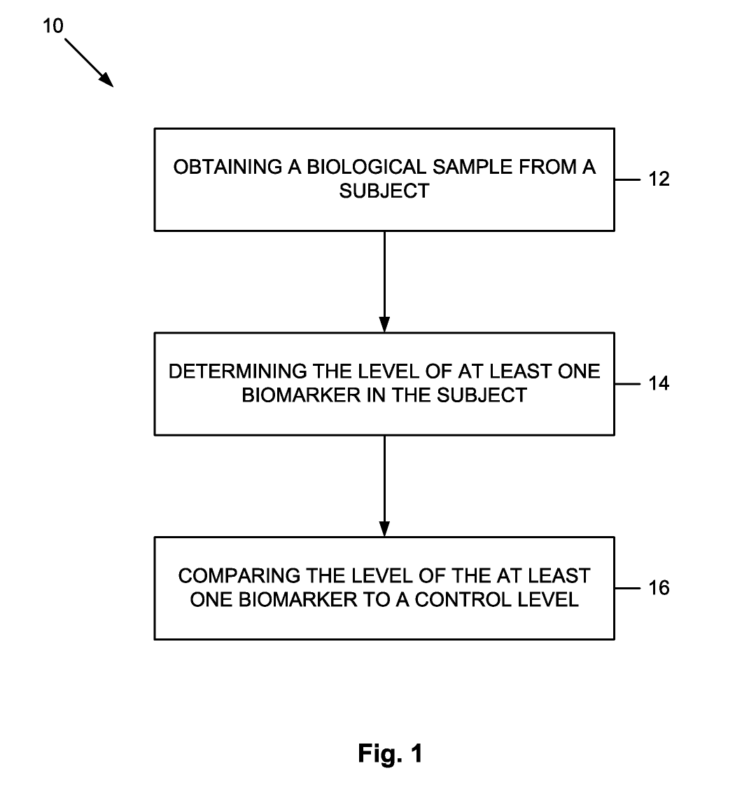

One aspect of the present disclosure is illustrated in FIG. 1 and includes a method 10 for detecting cancer (e.g., early stage cancer) in a subject. The method 10 generally includes diagnosing cancer (e.g., early stage cancer) by measuring the level of at least one specific biomarker present in a biological sample (e.g., human serum), and then comparing the detected biomarker level(s) to a control or normal reference level. The method 10 may be used for the detection and diagnosis of cancer (e.g., early stage cancer), especially in high risk populations. The method 10 may also be incorporated into a high-throughput screening method for testing large number of subjects, thereby enabling longitudinal screening throughout the lifetime of a subject to assess risk and detect cancer early on. The method 10 therefore has the potential to detect cancer progression prior to that detectable by conventional methods (e.g., computed tomography), which is critical to positive treatment outcome.

Cancer detectable by the method 10 can include any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys, pancreas and liver.

In one example of the present disclosure, cancer detectable by the method 10 can include lung cancer. As used herein, “lung cancer” can refer to any neoplastic modification affecting one of more cells present in the lung tissue. Exemplary non-limiting types of lung cancer can include small and non-small cell lung cancer, including squamous cell carcinoma, adenocarcinoma, bronchogenic, large cell carcinoma, carcinoid and mesothelioma.

Cancer detectable by the method 10 can further include early stage cancer. As used herein, “early stage cancer” can refer to those cancers that have been clinically determined to be organ-confined. Also included are tumors too small to be detected by conventional methods, such as X-rays for subjects with lung cancer. In one example of the present disclosure, early stage lung cancer can refer to stage I or II non-small-cell lung cancer in which the cancer is confined to the lung(s). In another example of the present disclosure, early stage lung cancer can refer to limited stage small-cell lung cancer in which the cancer is confined to its area of origin in the lung(s) and lymph nodes.

In another example, early stage prostate cancer can refer to stage 1 or stage 2 prostate cancer in which the cancer does not spread outside of the prostate.

In another example, early stage colon cancer can refer to stage 0, stage 1, or stage 2A colon cancer in which the cancer has not spread to nearby lymph nodes.

In another example, early stage renal cancer can refer to stage 1 or stage 2 renal cancer that is completely within the kidney(s).

Step 12 of the method 10 includes obtaining a biological sample from a subject. The biological sample could originate from anywhere within the body of the subject, for example, blood (e.g., serum/plasma), cerebral spinal fluid, bile, urine, stool, breath, saliva, or biopsy of any solid tissue including tumor, adjacent normal tissue, smooth and skeletal muscle, adipose tissue, liver, skin, hair, brain, kidney, pancreas, lung, colon, stomach, or other. While the term “serum” is used herein, those skilled in the art will recognize that plasma, whole blood, or a sub-fraction of whole blood may be used. In one example of the present disclosure, the biological sample can comprise about 200 μl of serum or plasma obtained from about 0.5 ml of blood.

When a blood sample is drawn from a subject, there are several ways in which the sample can be processed. The range of processing can be as little as none (i.e., frozen serum) or as complex as the isolation of a particular cell type. The most common and routine procedures involve the preparation of either serum or plasma from whole blood. All blood sample processing methods, including centrifugation and spotting of blood samples onto solid-phase supports, such as filter paper or other immobile materials, are also contemplated by the present disclosure.

Without wishing to be limiting, the processed blood or plasma sample described above may then be further processed to make it compatible with the methodical analysis technique(s) to be employed in the detection and measurement of certain biomarkers contained within the processed blood sample. The types of processing can range from as little as no further processing to as complex as differential extraction and chemical derivatization. Extraction methods may include sonication, soxhiet extraction, supercritical fluid extraction, accelerated solvent extraction, chromatography surfactant assisted extraction in common solvents, such as methanol, ethanol, chloroform, mixtures of methanol and chloroform, or organic solvents, such as ethyl acetate or hexane.

Following extraction of the biological sample, the level of at least one biomarker in the subject is determined at Step 14. In other words, Step 14 can be performed on a biological sample that was previously withdrawn from the subject. The determined level can be measured using any suitable quantitative unit (or units), in either log- or non-log scale, depending upon the type of assay(s) or technique(s) used to analyze the biological sample. For example, the determined level can be measured in terms of μmol/L, nmol/L, or metabolite peak value/internal standard peak value. The extracted biological sample may be analyzed using any suitable method (e.g., an in vitro or ex vivo method) known in the art, such as ultraviolet (UV) detection (e.g., UV-visible spectrophotometry), colorimetric assays, chemical titration, thermometric titration, using a fatty acid binding protein, spectrophotometry, calorimetric or fluorometric spectrophotometry, microplate readers, mini-scan analysis, chromatography (e.g., thin layer chromatography), free fatty acid assay kits, free fatty acid quantification kits, high-performance liquid chromatography (HPLC), radiochemical assays, antibody-based assays, fluorescence, enzymatic assays, fluorogenic assays, phospholipid assay kits, and the like. For example, and without wishing to be limiting, the biological sample can be amenable to analysis on essentially any mass spectrometry platform (e.g., tandem mass spectroscopy), either by direct injection or following chromatographic separation. Typical mass spectrometers are comprised of a source that ionizes molecules within the sample, and a detector for detecting the ionized molecules or fragments of molecules. Non-limiting examples of common sources include electron impact, electrospray ionization (ESI), atmospheric pressure chemical ionization, atmospheric pressure photo ionization, matrix assisted laser desorption ionization, surface enhanced laser desorption ionization, and derivations thereof. Common mass separation and detection systems can include quadrupole, quadrupole ion trap, linear ion trap, time-of-flight, magnetic sector, ion cyclotron, Orbitrap, and derivations and combinations thereof.

In one example of the present disclosure, the biological sample is analyzed to determine the level of at least one biomarker using on-line ESI tandem mass spectroscopy in the positive ion mode with multiple reaction monitoring including the molecular cation [MH]+ and the m/z 184 daughter phosphocholine ion or negative ion mode and multiple reaction monitoring (MRM) using characteristic parent→daughter ion transitions.

The at least one biomarker detectable by the method 10 can include any carboxylic acid-containing molecule. For example, the at least one biomarker can include a free fatty acid, a phospholipid, or a metabolite thereof. The at least one free fatty acid can be selected from the group of oleic acid, stearic acid, arachidonic acid (AA), palmitic acid, linoleic acid (LA), eicosapentanoic acid and docosahexanoic acid. In one example of the present disclosure, the at least one free fatty acid can comprise a hydroxyeicosatetraenoic acid (HETE), such as 15-HETE, 12-HETE, 11-HETE or 5-HETE. In another example of the present disclosure, the at least one free fatty acid can comprise a hydroxyoxyoctadecadienoic acid (HODE), such as 13-HODE and 9-HODE. HETE and HODE promote tumor development, progression, and metastasis in cell-culture, tissue and animal studies.

The at least one phospholipid can comprise a hydrophobic molecule including one or more phosphorus groups. For example, a phospholipid can comprise a phosphorus-containing group and saturated or unsaturated alkyl group, optionally substituted with OH, COOH, oxo, amine, or substituted or unsubstituted aryl groups. Non-limiting phospholipids that can be detected by the method include phosphatidic acid, phosphatidylglycerol, phosphatidylcholine (PC), lysophosphatidylcholine (LPC), phosphatidylethanolamine, phosphatidylinositol, phosphatidylserine, Lyso-platelet activating factor (PAF), and oxidized phospholipids (e.g., Az-PAF, Az-PC, oxidized phospholipids hydroxyoctadecadienoyl phosphatidylcholine (HODE-PC) and hydroperoxyoctadecadienoyl phosphatidylcholine (HpODE-PC)).

In one example of the present disclosure, the at least one biomarker can comprise LPC. LPC molecules detectable by the method can have varying acyl chain lengths (e.g., about 12 to about 18 carbon atoms). In another example of the present disclosure, the at least one biomarker can comprise LPC-C16.

The level of one, two, three, or more biomarkers can be determined at Step 14. The detected biomarkers can be the same or different. For example, the level of two free fatty acids (e.g., 13-HODE and 9-HODE) can be determined at Step 14. Additionally or alternatively, the level of four free fatty acids (e.g., 15-HETE, 12-HETE, 11-HETE, and 5-HETE) can be detected at Step 14. Additionally or alternatively, the level of one or more phospholipids (e.g., LPC-C16 or HODE-PC) can be determined at Step 14.

At Step 16, the detected level of at least one biomarker is compared to the level of at least one biomarker in a control. The control can be any suitable reference sample for the particular cancer. For example, and without wishing to be limiting in any manner, the control may be a sample from a control subject, i.e., a subject not suffering from cancer and with or without a family history of cancer. In some instance, the control may be a sample obtained from a subject clinically diagnosed with a cancer but before a medical intervention. In other instances, the control may be a sample obtained from a subject soon after performing a medical intervention on the subject. In still other instances, a suitable control can be established by assaying a large sample of subjects that do not have a particular cancer and using a statistical model to obtain a control value (standard value).

As would be understood by a person of skill in the art, more than one control may be used for comparison to the data obtained at Step 16. For example, and without wishing to be limiting, the control may be a first reference sample obtained from a non-cancer control subject. In the case of monitoring a subject's change in cancer state, the control may include one or more samples obtained at an earlier time period (or periods) either pre-therapy or during therapy to compare the change in cancer state as a result of therapy.

Following comparison of the control level with the detected or determined level of the at least one biomarker, a diagnosis can be made (e.g., by a medical professional) as to whether the subject has cancer (e.g., early stage cancer). In some instances, a detected level of at least one biomarker that is increased or elevated as compared to a control level can be indicative of cancer (e.g., early stage cancer) in the subject. For example, a level of at least one biomarker that is at least about 2-fold higher than the control level can be indicative of cancer (e.g., early stage cancer) in the subject. In one example of the present disclosure, a detected level of at least one free fatty acid (e.g., a HETE or HODE) that is about 2-fold higher than the control level may be indicative of cancer (e.g., early stage cancer) in the subject. In another example of the present disclosure, a detected level of 15-HETE, 12-HETE, 11-HETE, and/or 5-HETE that is about 8-fold to about 25-fold higher (e.g., about 20-fold higher) than the control level may be indicative of cancer (e.g., early stage non-small-cell lung cancer). In another example of the present disclosure, a detected level of 13-HODE and/or 9-HODE that is about 12-fold to about 24-fold higher (e.g., about 20-fold higher) than the control level may be indicative of cancer (e.g., early stage non-small-cell lung cancer).

In another example of the present disclosure, a detected level (e.g., nmol/L) of 15-HETE that is about 22-fold to about 27-fold higher (e.g., about 25-fold higher) than the control level may be indicative of cancer (e.g., early stage non-small-cell lung cancer).

In another example of the present disclosure, a detected level (e.g., nmol/L) of 12-HETE that is about 6-fold to about 10-fold higher (e.g., about 8-fold higher) than the control level may be indicative of cancer (e.g., early stage non-small-cell lung cancer).

In another example of the present disclosure, a detected level (e.g., nmol/L) of 11-HETE that is about 12-fold to about 16-fold higher (e.g., about 14-fold higher) than the control level may be indicative of cancer (e.g., early stage non-small-cell lung cancer).

In another example of the present disclosure, a detected level (e.g., nmol/L) of 5-HETE that is about 19-fold to about 23-fold higher (e.g., about 21-fold higher) than the control level may be indicative of cancer (e.g., early stage non-small-cell lung cancer).

In another example of the present disclosure, a detected level (e.g., nmol/L) of 13-HETE that is about 10-fold to about 15-fold higher (e.g., about 12-fold higher) than the control level may be indicative of cancer (e.g., early stage non-small-cell lung cancer).

In another example of the present disclosure, a detected level (e.g., nmol/L) of 9-HODE that is about 22-fold to about 26-fold higher (e.g., about 24-fold higher) than the control level may be indicative of cancer (e.g., early stage non-small-cell lung cancer).

In another example of the present disclosure, a detected level (e.g., nmol/L) of AA that is about 2-fold to about 6-fold higher (e.g., about 3-fold higher) than the control level may be indicative of cancer (e.g., early stage non-small-cell lung cancer).

In another example of the present disclosure, a detected level (e.g., nmol/L). of LA that is about 10-fold to about 15-fold higher (e.g., about 12-fold higher) than the control level may be indicative of cancer (e.g., early stage non-small-cell lung cancer).

In one example of the present disclosure, a detected level (e.g., μmol/L) of at least one phospholipid (e.g., LPC) that is about 2-fold higher than the control level may be indicative of cancer (e.g., early stage cancer) in the subject. For instance, a detected level (e.g., μmol/L) of LPC-C16 that is about 2-fold to about 4-fold higher than the control level may be indicative of cancer (e.g., early stage non-small-cell lung cancer).

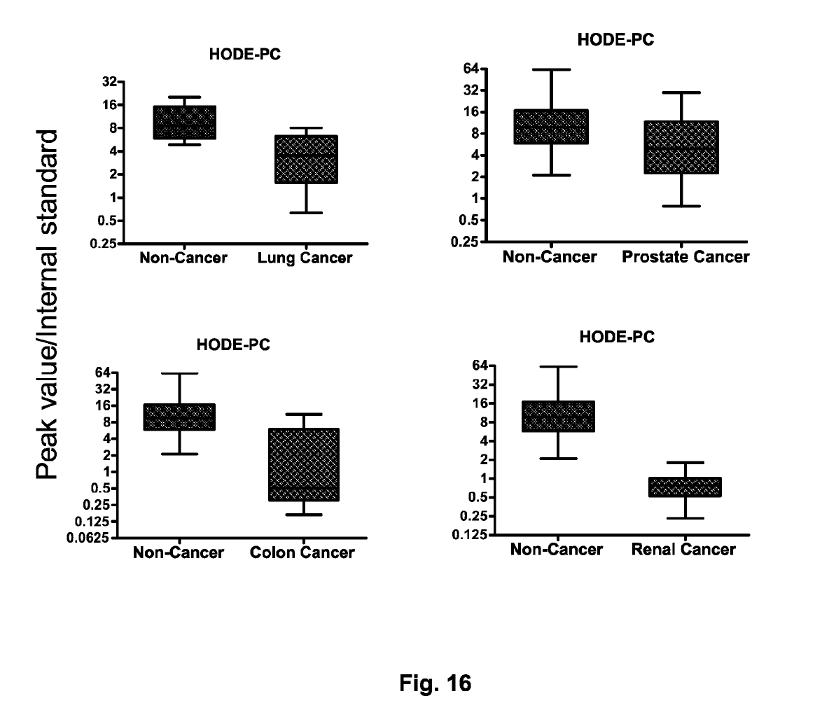

In another aspect, a detected level of at least one biomarker that is decreased or less than a control level can be indicative of cancer (e.g., early stage cancer) in the subject. In some instances, the detected biomarker can be oxidized phospholipid hydroxyoctadecadienoyl phosphatidylcholine (HODE-PC). As shown in FIG. 16, it was discovered that median HODE-PC levels were significantly decreased in patients with lung, renal, prostate and colon cancer, indicating that blood lipoprotein-associated phospholipase A2 (Lp-PLA2) activity may be increased in cancer patients. LpPLA2 activity may, in some instances, serve as a cancer biomarker and a potential anti-cancer therapy target. The method of HODE-PC measurement was the same as other phospholipids (e.g., LPC) as described in Example 1 below.

In one example, a level of at least one biomarker (e.g., HODE-PC) that is at least about 2-fold to about 6-fold (e.g., 3-fold) less than the control level can be indicative of cancer (e.g., early stage lung cancer) in the subject. In another example, a level of at least one biomarker (e.g., HODE-PC) that is at least about 10-fold to about 20-fold (e.g., 15-fold) less than the control level can be indicative of cancer (e.g., early stage colon cancer) in the subject. In another example, a level of at least one biomarker (e.g., HODE-PC) that is at least about 2-fold to about 4-fold less than the control level can be indicative of cancer (e.g., early stage prostate cancer) in the subject. In another example, a level of at least one biomarker (e.g., HODE-PC) that is at least about 8-fold to about 12-fold (e.g., 9-fold) less than the control level can be indicative of cancer (e.g., early stage renal cancer) in the subject.

If the diagnosis made following Step 16 is positive for cancer (e.g., early stage cancer), one skilled in the art will appreciate how to initiate and tailor an appropriate therapy regimen for the particular type of cancer. The method 10 can be used as annual testing for an older population of subjects (e.g., greater than 50 years-old), for a younger population (e.g., less than 40 years-old) that have a family history or high risk factors for cancer, and/or for routine testing of subjects at any age who are suspected of having cancer. Advantageously, the method 10 provides a reliable, risk-free test (e.g., blood test) for determining one or more biomarkers in a subject that may be used to detect the presence of cancer (e.g., at an early stage) and thereby improve outcomes by detecting cancer early enough for a medical intervention (e.g., surgery) to provide a good chance of cure.

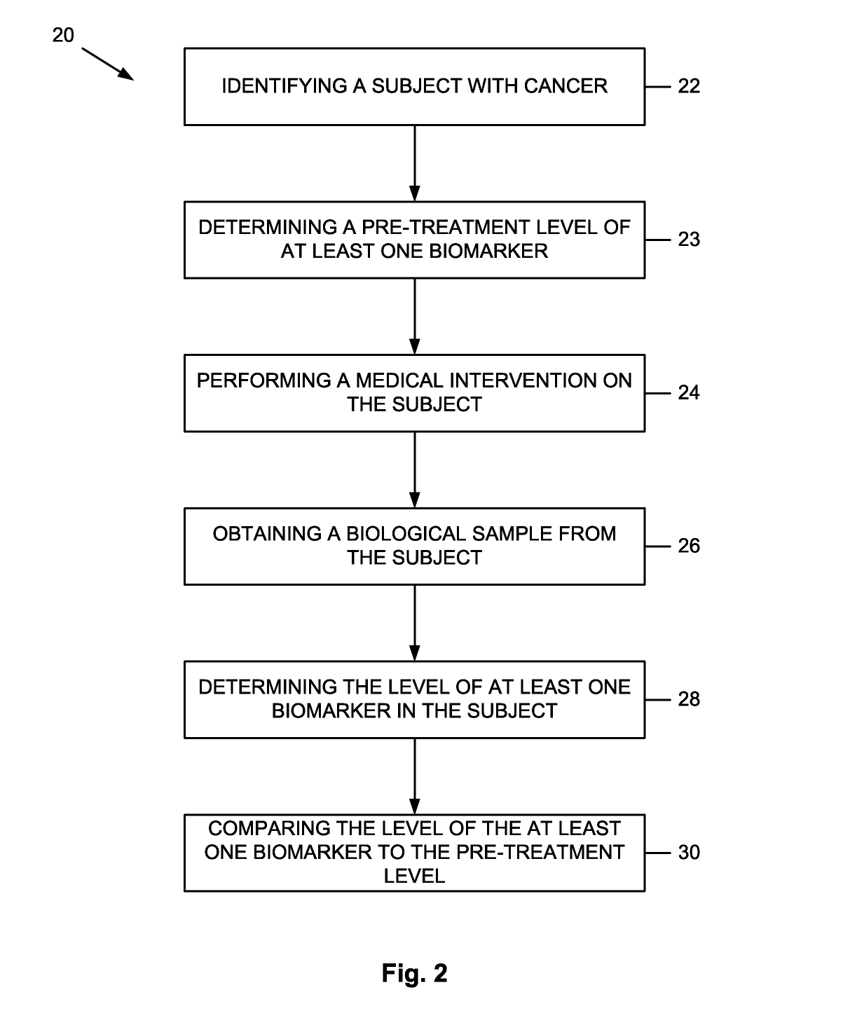

Another aspect of the present disclosure is illustrated in FIG. 2 and includes a method 20 for predicting the prognosis of a subject with cancer following a medical intervention to treat the cancer. One step of the method 20 includes identifying a subject with cancer (Step 22). One skilled in the art will appreciate that there are numerous cancer detection tests that can be used to detect cancer, examples of which include, but are not limited to, anti-malignin antibody screen test, various cancer biomarker tests (e.g., alpha fetoprotein, CA 15.3, carcinoembryonic antigen, etc.), CBC blood test, various types of microscopy, PET scanning, CT scanning, ultrasound or sonogram, MRI, thermography, and T/Tn antigen testing. To detect certain types of lung cancer, for example, any one or combination of a sputum cytology analysis, a PET scan, a CT scan, X-rays, CA125, DR-70 and the T/Tn antigen test can be used. In one example of the present disclosure, Step 22 of the method 20 can include detecting early stage cancer (e.g., non-small-cell lung cancer) in a subject according to the method described above.

Once the subject has been diagnosed with cancer, a medical intervention is performed on the subject to treat the cancer at Step 24. The type of medical intervention performed will depend upon the type of cancer. Types of medical interventions that may be employed to treat the cancer can include surgery, chemotherapy, radiotherapy, other types of surgical procedures (e.g., bone marrow transplantation and peripheral blood stem cell transplantation), and combinations thereof. Where the cancer includes non-small-cell lung cancer, for example, a surgical intervention can be performed to resect one or more tumors identified at Step 22. In one example of the present disclosure, the medical intervention can substantially eliminate the cancer. By “substantially eliminate”, it is meant that the presence of a cancer, as measurable by one or more conventional diagnostic assays, is substantially reduced or entirely eliminated. For example, “substantially eliminated” can mean that the tumor load in a subject has been reduced by about 50%, about 60%, about 70%, about 80%, about 90%, or about 100%.

Following the medical intervention, one or more biological samples can be obtained from the subject at Step 26. The biological sample(s) can be obtained in an identical or similar manner as described at Step 12 (FIG. 1) above. For example, a volume of serum (e.g., about 200 pi) can be collected from the subject. The biological sample(s) can be obtained at a desired period of time following the medical intervention. For example, the biological sample(s) can be obtained at less than 6 hours, about 6 hours, about 12 hours, about 24 hours, about 2 weeks, monthly, or more following the medical intervention.

At Step 28 (FIG. 2), the level of at least one biomarker in the subject can be determined in a similar or identical manner as described at Step 14 (FIG. 1) above. For example, on-line ESI tandem mass spectroscopy can be used to detect or determine the level of at one least free fatty acid (e.g., 15-HETE, 12-HETE, 11-HETE, 5-HETE, 13-HODE, 9-HODE, AA or LA) or phospholipid (e.g., LPC-C16 or HODE-PC) in the subject.

After determining the level of the at least one biomarker, the detected level of the biomarker(s) is compared to a pre-treatment level of at least one biomarker at Step 30 (FIG. 2). Step 30 can be performed in a similar or identical manner as Step 16 (FIG. 1) above. For example, the level of the detected biomarker(s) can be compared to a pre-treatment level that corresponds to the level of the biomarker(s) prior to the medical intervention. In such instances, a biological sample can be obtained at Step 23 and then analyzed to determine the level of at least one biomarker prior to the medical intervention on the subject. It will be appreciated that the level of the detected biomarker(s) can alternatively or additionally be compared to a control level (as described above). Upon comparing the detected level of the biomarker(s) with the pre-treatment level, a decreased level of the detected biomarker(s) as compared to the pre-treatment level may be indicative of a favorable prognosis in the subject. As used herein, the term “favorable prognosis” can refer to an increased likelihood that a subject will remain cancer-free (e.g., no recurrences or metastasis) for a period of time, such as at least one, two, three, four, five years or more of initial diagnosis of cancer.

In one example of the present disclosure, a biological sample can be obtained from a subject with early stage cancer (e.g., non-small-cell lung cancer) about 6-24 hours to 2 weeks after a medical intervention (e.g., tumor resection) and then assayed to determine the level of one or more phospholipids (e.g., LPC-C16). A detected level (e.g., μmol/L) of the phospholipid(s) that is/are decreased at least about 2-fold as compared to the pre-treatment level may be indicative of a favorable prognosis.

In another example of the present disclosure, a biological sample can be obtained from a subject with early stage cancer (e.g., non-small-cell lung cancer) about 24 hours to about a month after a medical intervention (e.g., tumor resection), and then assayed to determine the level of one or more free fatty acids (e.g., HETEs and/or HODEs). A detected level (e.g., nmol/L) of the one or more free fatty acid that is decreased at least about 3-fold (e.g., about 3-fold to about 10-fold) as compared to the pre-treatment level may be indicative of a favorable prognosis.

Where the level of the detected biomarker(s) is/are the same as, or higher than, the pre-treatment level, a determination may be made that the subject has an unfavorable or poor prognosis. A “poor prognosis” as used herein can mean an expectation of a recurrence or metastasis within one, two, three, four, or five years of initial diagnosis of cancer. A poor prognosis may also indicate that a tumor is relatively aggressive (while a favorable prognosis may indicate that a tumor is relatively nonaggressive).

It will be appreciated that the prognosis results obtained according to the method 20 (FIG. 2) can also be correlated to, or serve as a basis for, a risk classification of the subject(s). As used herein, “risk classification” can mean the level of risk or the prediction that a subject will experience a particular clinical outcome. A subject may be classified into a risk group or classified at a level of risk based on the predictive method of the present disclosure. A “risk group” can refer to a group of subjects with a similar level of risk for a particular clinical outcome.

Following Step 30, a treatment decision can be for a subject deemed to have a poor prognosis. For example, one skilled in the art will appreciate that a treatment regimen can be implemented that is likely to induce complete remission and prevent relapse, or any treatment that a medical practitioner may deem appropriate for a subject with a poor prognosis.

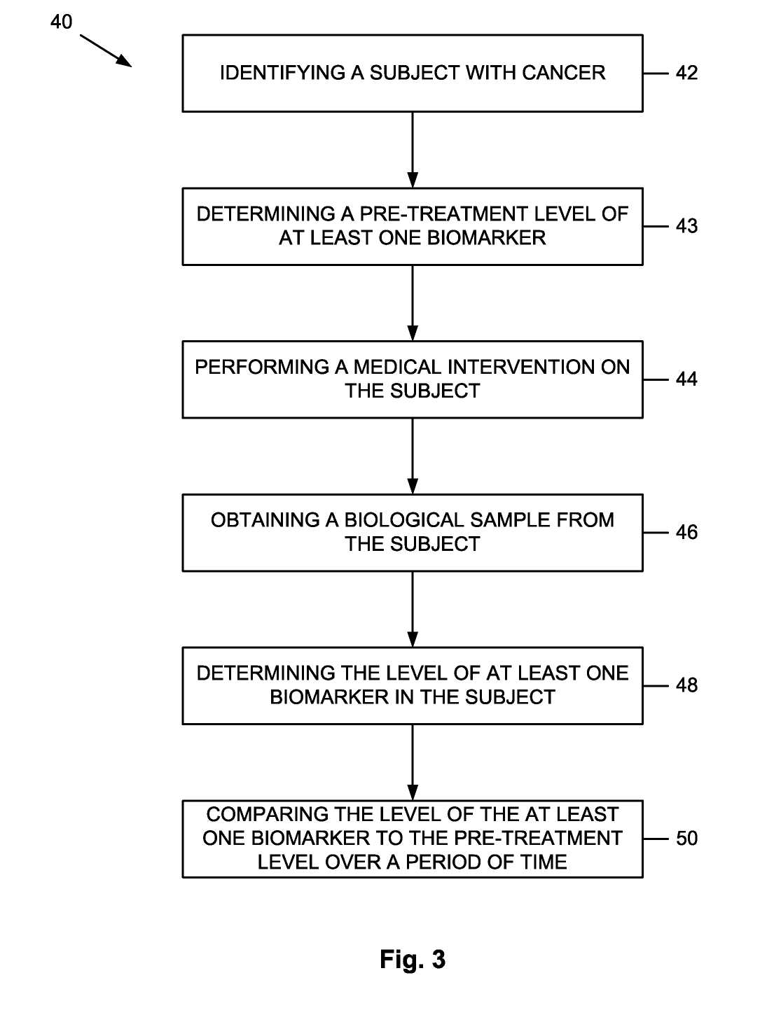

Another aspect of the present disclosure is illustrated in FIG. 3 and includes a method 40 for detecting cancer recurrence in a subject following a medical intervention to treat the cancer. The method 40 can begin by identifying a subject with cancer at Step 42. Step 42 can be performed in an identical or similar manner as described above in Step 22 (FIG. 2). For example, a subject having early stage cancer, such as non-small-cell lung cancer can be identified according to the method described above.

Once the subject has been diagnosed with cancer, a medical intervention is performed on the subject to treat the cancer (Step 44) (FIG. 3). Step 44 can be performed in a similar or identical manner as described in Step 24 (FIG. 2) above. For example, the subject can be treated with one or a combination of known medical interventions, such as surgery (e.g., tumor resection), chemotherapy, radiation therapy, and combinations thereof.

Following the medical intervention, one or more biological samples can be obtained from the subject at Step 46 (FIG. 3). The biological sample(s) can be obtained in an identical or similar manner as described in Step 26 (FIG. 2) above. For example, a volume of serum (e.g., about 200 μl) can be collected from the subject monthly. The biological sample(s) can be obtained at any desired period of time following the medical intervention.

At Step 48 (FIG. 3), the level of at least one biomarker can be determined in a similar or identical manner as described in Step 28 (FIG. 2) above. For example, on-line ESI tandem mass spectroscopy can be used to detect or determine the level (e.g., nmol/L or metabolite peak value/internal standard peak value) of at one least free fatty acid (e.g., 15-HETE, 12-HETE, 11-HETE, 5-HETE, 13-HODE, 9-NODE, AA or LA) or phospholipid (e.g., LPC-C16 or HODE-PC) in the subject.

After determining the level of the at least one biomarker, the detected level of the biomarker(s) is/are compared to a pre-treatment level of at least one biomarker (Step 50) (FIG. 3). Step 50 can be performed in a similar or identical manner as Step 30 (FIG. 2) above. For example, the level of the detected biomarker(s) can be compared to a pre-treatment level soon after the medical intervention. In such instances, a biological sample can be obtained at Step 43 and then analyzed to determine the level of at least one biomarker prior to the medical intervention on the subject. It will be appreciated that the level of the detected biomarker(s) can alternatively or additionally be compared to a control level (as described above). The level(s) of the biomarker(s) can be monitored over a period of time, such as at regular intervals (e.g., once every month, once a year, once every two years) to determine whether the level(s) of the biomarker(s) change (e.g., decrease or increase) over time. Where the level(s) of the detected biomarker(s) decrease (e.g., to a normal or control level and remain) over time, for example, a determination may be made that the subject has a favorable prognosis.

Alternatively, where the level(s) of the detected biomarker(s) is/are the same as, or increased again over time, a determination may be made that the subject has an unfavorable or poor prognosis or cancer recurrence.

Following Step 50 (FIG. 3), a treatment decision can be made for a subject deemed to have a poor prognosis or cancer recurrence. For example, one skilled in the art will appreciate that a treatment regimen can be implemented that is likely to induce complete remission and prevent relapse, or any treatment that a medical practitioner may deem appropriate for a subject with a poor prognosis.

Without wishing to be limiting in any way, it will be appreciated that the present disclosure can be carried out, at least in part, with the assistance of a computer that includes a computer-readable medium for storing and/or processing data. In such instances, the computer may be integrated with an instrument (e.g., a mass spectroscopy unit) used to perform the analysis, or it may be a separate computer adapted to receive data output from the instrument according to the knowledge and skill of those in the art. The analyzing step (a) will typically be carried out using the instrument, such as a mass spectrometer, and the comparing step (b) carried out using the computer or other processing means programmed to receive the accurate mass intensity data or quantifying data from the instrument and perform the calculations required to identify an increase or decrease in the level of the one or more biomarkers in a biological sample. This data from step (b) may be output for use by an individual trained to identify the noted increase or decrease and make the diagnosis of step (c) or, alternatively, the computer or processing means may be further programmed to generate an output of a diagnosis. In the latter case, the output may comprise a positive or negative diagnosis and/or prognosis factor, and may optionally include additional details including, but not limited to, statistical data, threshold data, patient data and other details. The data may be output to a display, such as a monitor, to a printer for generating a copy of the details of diagnosis and/or prognosis, to a data receiving center or directly to a service provider, or in any other way as would be understood by one skilled in the art.

### Kits

In another aspect, kits for practicing the present disclosure are included. A kit can include one or more carriers, each of which is suited for containing one or more container means, and instructions for carrying out one or more of the methods described herein. In some instances, container means can include vials, tubes, bottles, dispensers, and the like, capable of holding one or more reagents needed to practice the present disclosure. In view of the description provided herein of the present disclosure, those of skill in the art can readily determine the apportionment of the necessary reagent(s) among the container mean(s).

Instructions for kits of the present disclosure can be affixed to packaging material or can be included as a package insert. While the instructions are typically written or printed materials, they are not limited to such. Any medium capable of storing such instructions and communicating them to an end user is contemplated by the present disclosure. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions.

In one example, a kit can include instructions for carrying out any of the methods 10 (FIG. 1), 20 (FIGS. 2), and 40 (FIG. 3) described above. In other instances, the kit can include at least one carrier means containing an agent capable of detecting the level of at least one biomarker (e.g., a phospholipid, a free fatty acid, or a metabolite thereof) in a biological sample. Agents capable of detecting the level of the at least one biomarker are known in the art and can include, for example, antibodies, fluorescent probes, etc. The kit can additionally or optionally include reagents for assays capable of detecting the level of at least one biomarker, such as those needed for a colorimetric, fluorometric, or an enzymatic assay. In another example, the kit can additionally or optionally include reagents needed to detect the level of at least one biomarker using mass spectroscopy, such as internal standards and reagents (e.g., solvents) for separating and/or extracting biomarkers from a biological sample (e.g., using column chromatography). It will be appreciated that a kit can include an additional container having one or more components (e.g., a separation means, such as a chromatography column) when the kit is configured for detection of biomarkers using mass spectroscopy. It will also be appreciated that a kit can include computer software for comparing the mass spectra of one or more biomarkers with the mass spectra of a control and calculating the level of the biomarker(s) in a biological sample.

In another example, a kit can comprise: at least one agent capable of detecting the level of at least one biomarker in a biological sample previously withdrawn from the subject; and instructions for use of the kit to detect cancer, cancer recurrence, or the prognosis of the subject following a medical intervention, to treat cancer in the subject by comparing a detected level of the at least one biomarker to a control. In some instances, a detected level of the at least one biomarker that is increased (e.g., about 2-fold greater) as compared to the level of the control is indicative of cancer in the subject. In other instances, a detected level of the at least one biomarker that is decreased as compared to the level of the control is indicative of cancer in the subject. In other instances, a detected level of the at least one biomarker that is decreased as compared to a pre-treatment level of at least one biomarker, following a medical intervention to treat the subject, is indicative of a positive prognosis in a subject with cancer. In further instances, a detected level of the at least one biomarker that is at least about 2-fold higher than a pre-treatment level of at least one biomarker over a period of time is indicative of cancer recurrence in the subject following a medical intervention to treat the cancer.

In another example, a kit can comprise: one or more reagents to facilitate detection of at least one biomarker in a biological sample previously withdrawn from a subject; and instructions for use of the kit to detect cancer, cancer recurrence, or the prognosis of the subject following a medical intervention, to treat cancer in the subject by comparing a detected level of the at least one biomarker to a control. In some instances, a detected level of the at least one biomarker that is about 2-fold greater than the level of the control is indicative of cancer in the subject. In other instances, a detected level of the at least one biomarker that is decreased as compared to a pre-treatment level of at least one biomarker is indicative of a positive prognosis in a subject with cancer following a medical intervention to treat the subject. In further instances, a detected level of the at least one biomarker that is at least about 2-fold higher than a pre-treatment level of at least one biomarker over a period of time is indicative of cancer recurrence in the subject following a medical intervention to treat the cancer.

Examples of reagents to facilitate detection of at least one biomarker in a biological sample can include internal standards and solvents (e.g., chloroform/methanol) capable of extracting biomarkers from a biological sample, which are also compatible with mass spectroscopy.

Such kits can additionally or optionally include a biological sample collection means, such as a syringe, scalpel, swab, tweezers, or the like.

The following examples are for the purpose of illustration only and are not intended to limit the scope of the claims, which are appended hereto.

### EXAMPLE 1

### Methods

With IRB approval and informed consent, we enrolled 45 patients age 18-85 years-old with non-small-cell lung cancer that were scheduled for potentially curative tumor resection. We also enrolled 20 patients without known cancer who were scheduled for spine surgery.

Demographic and morphometric characteristics were recorded, as were anesthetic and surgical details. Venous blood was sampled before surgery, and again 6 and 24 hours postoperatively. Samples were centrifuged at 3000 times G, and the resulting serum was frozen at −70° C. until assayed.

200 μl of serum from each patient was used for phospholipid and free fatty acid extraction. Each of the serum samples was added with an equal amount of 4D-PAF and 8D-15-HETE (Cayman Chemical, Ann Arbor, Mich.) as internal standards for phospholipids or free fatty acids, respectively. The phospholipids and free fatty acids were extracted first using chloroform/methanol, and then further isolated by column chromatography. Fractions of phospholipids and free fatty acids were dried by liquid nitrogen and dissolved in 200 μl of 85% of menthol or 50% methanol in HPLC water, respectively.

Mass spectrometric analyses for phospholipids were performed on-line using electrospray ionization tandem mass spectrometry in the positive ion mode with multiple reaction monitoring using the molecular cation [MH]+ and the m/z 184 daughter phosphocholine ion. For free fatty acid analysis, negative-ion mode with multiple reaction monitoring of parent and individual daughter ions of oxidized and unoxidized fatty acids used the m/z transitions: 5-HETE (319→115); 8-HETE (319→155); 9-HETE (319→151); 11-HETE (319→3167); 12-HETE (319→179); 15-HETE (319→175); 9-NODE (295→4171); 13-RODE (295→195); arachidonate (303→259); and linoleate (279→261).

All analyses were conducted by an investigator blinded to patient status and sample time.

### Results

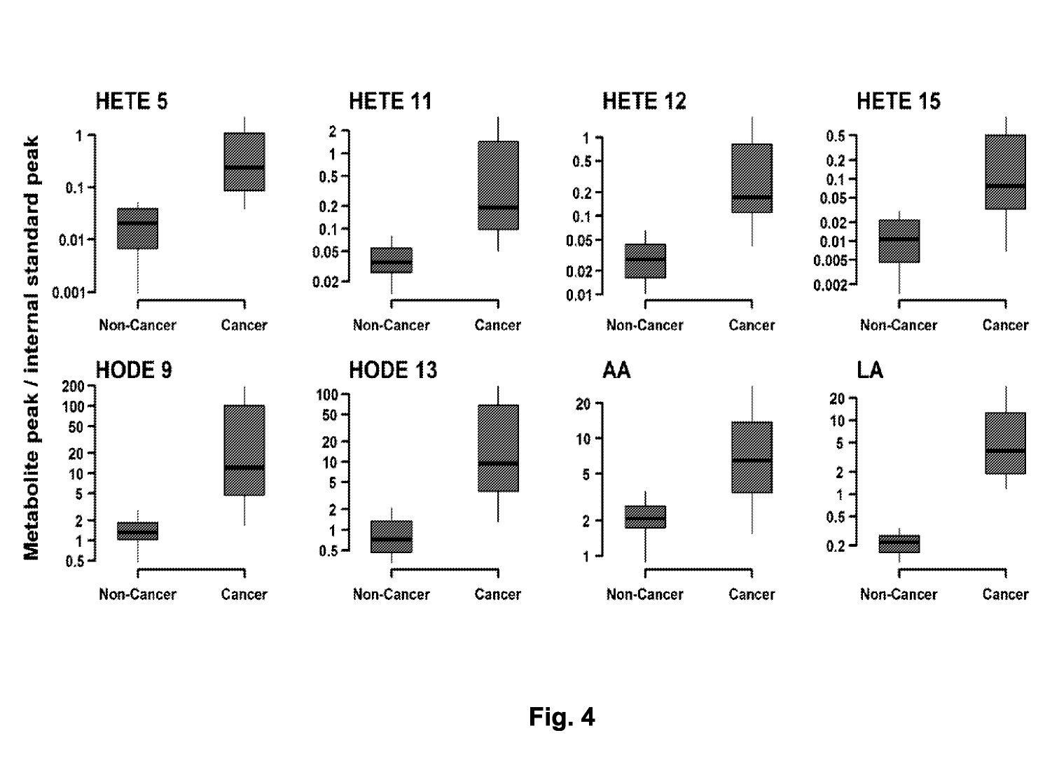

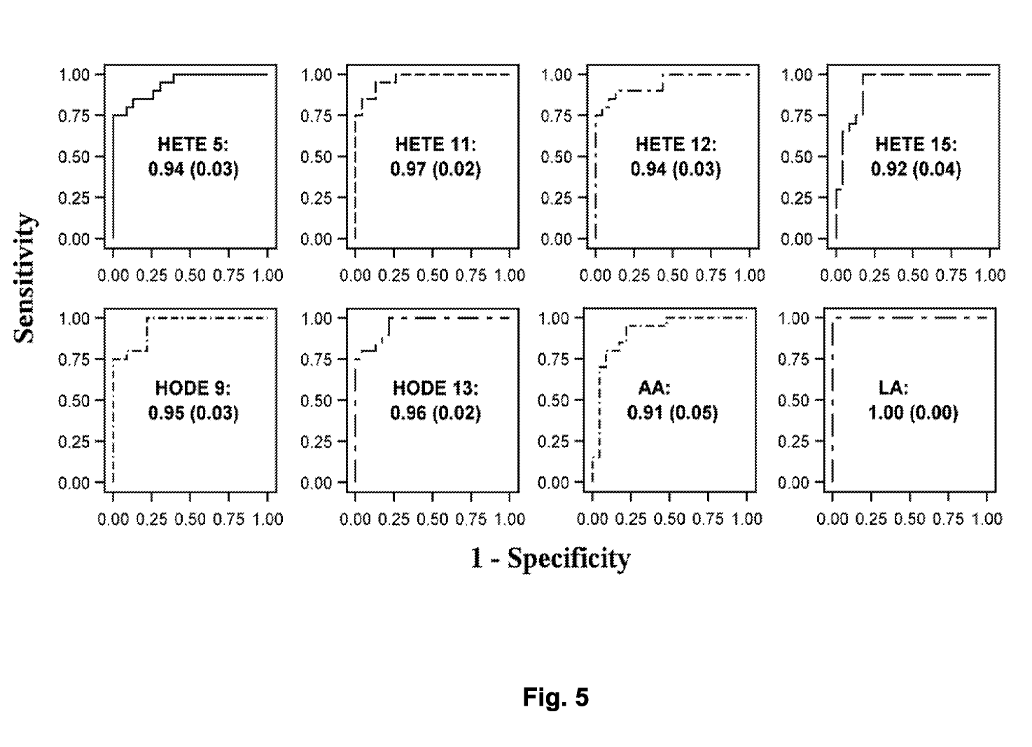

Serum oxidized free fatty acids linoleic acid (LA), arachidonic acid (AA), and their metabolites hydroxyeicosatetraenoic acids (HETEs, 5-, 11-, 12-, 15-HETE) and hydroxyoctadecadienes (HODEs, 9-, 13-HODE) were about 20-fold greater in patients with lung cancer than in surgical patients without cancer. There was little overlap between values in the cancer and non-cancer patients, and all differences were highly statistically significant (FIG. 4). As shown in FIG. 4, for example, median free fatty acid metabolite values were 10, 3, 17, 10, 8, 11, 14, and 17 times as large in cancer patients than controls for LA, AA, HETE-5, HETE-11, HETE-12, HETE-15, HODE-9 and HODE-13, respectively. Receiver Operating Characteristic curve for each free fatty acid metabolite at base line was typically 0.92-0.97 (FIG. 5).

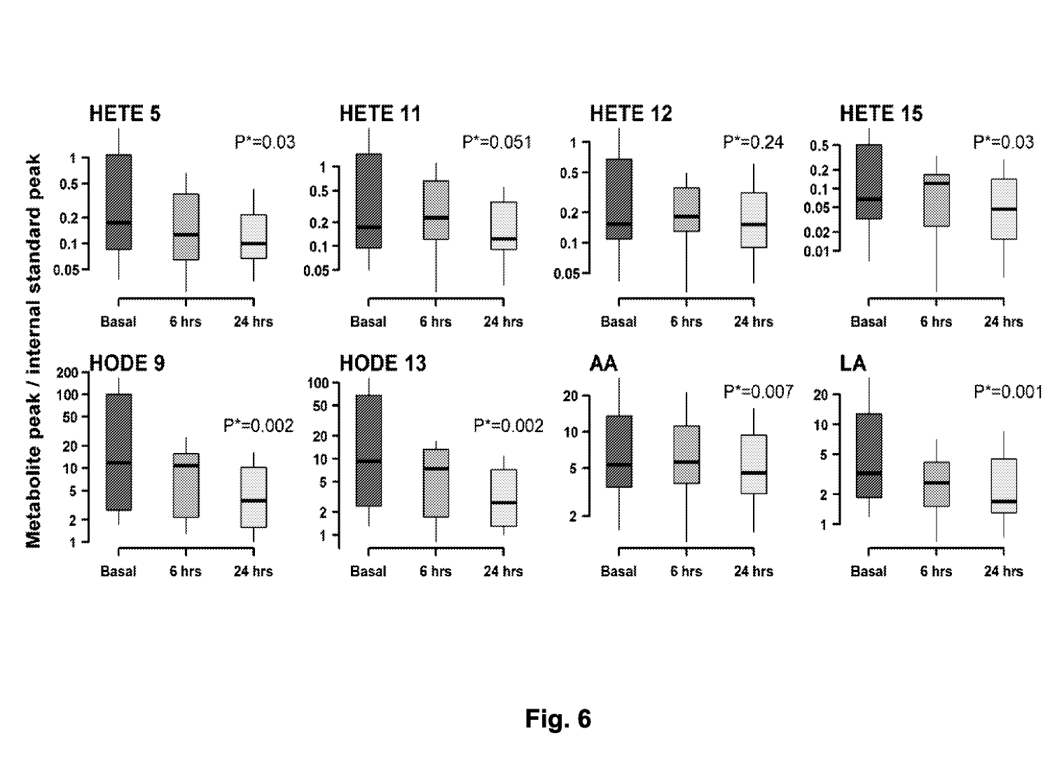

After tumor resection, concentrations of free fatty acids LA, AA, hydroxyeicosatetraenoic acids and hydroxyoctadecadienes decreased substantially in just 24 hours (FIG. 6). The percentage decrease from baseline to 24 hours was significant (p<0.05) for all metabolites (Mann-Whitney test) in lung cancer patients.

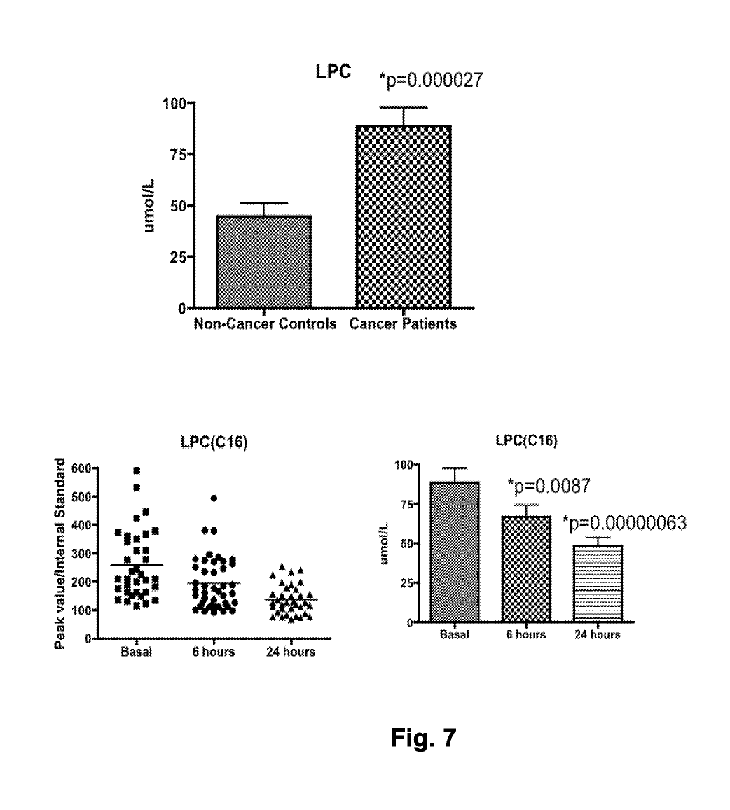

Serum phospholipid lysophosphatidylcholine (LPC-C16) concentrations were 2-3-fold greater in lung cancer patients and significantly decreased as short as 6 hours after tumor was surgically removed (FIG. 7).

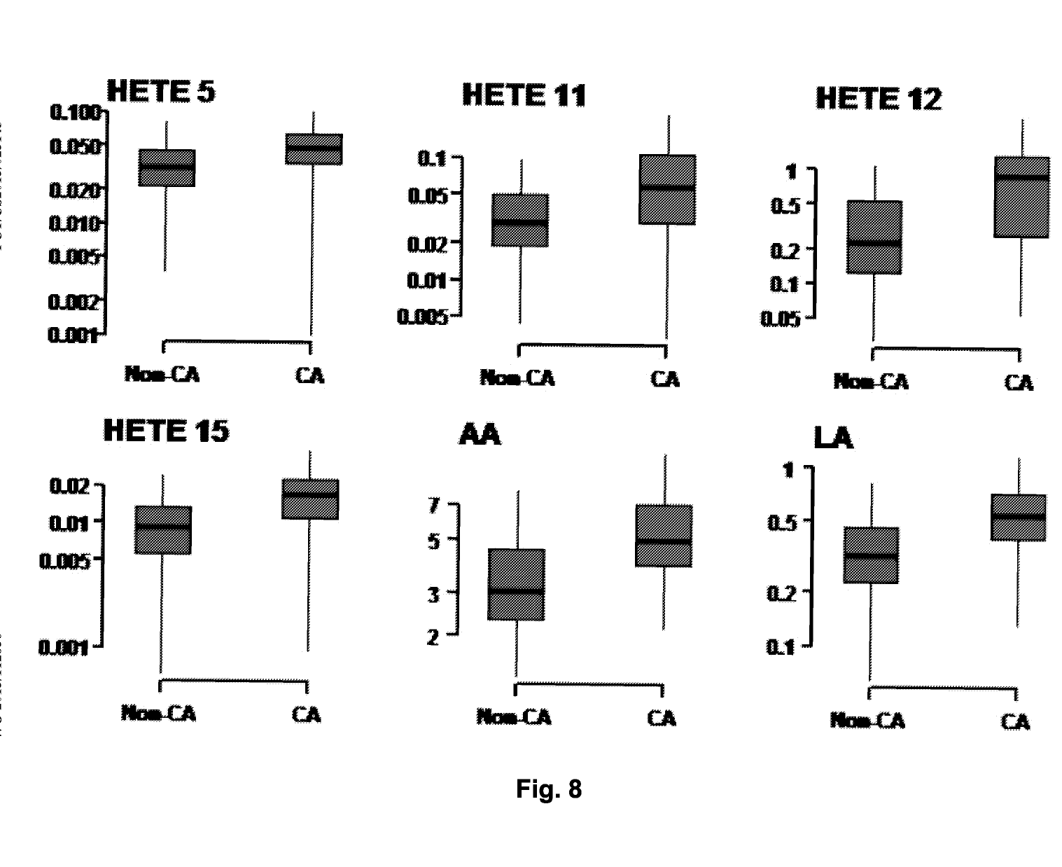

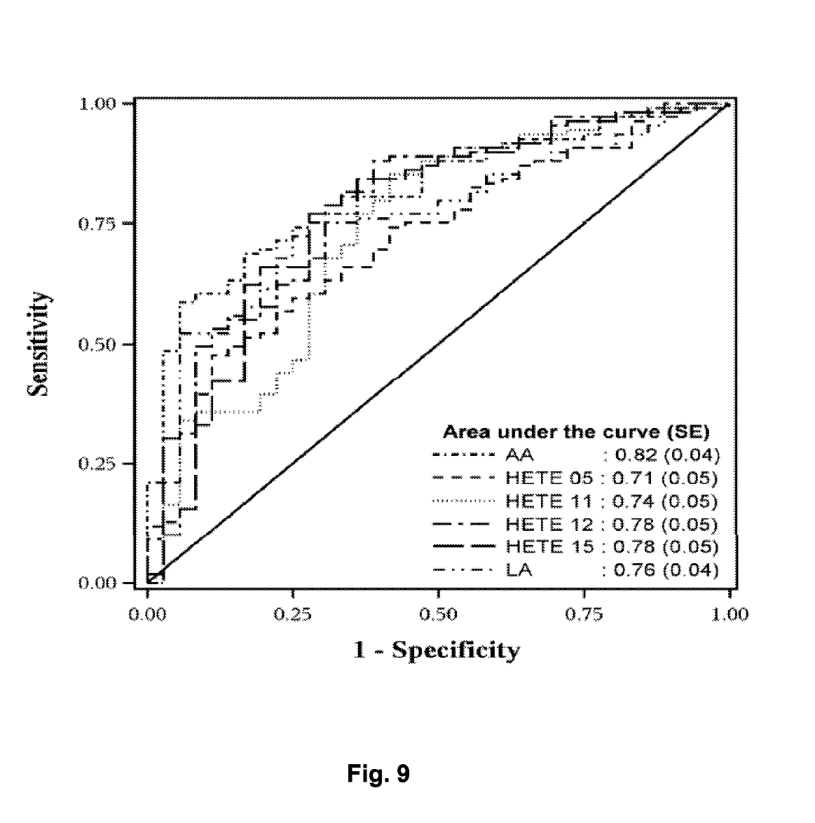

In a second study, the results of which are shown in FIGS. 8-9 and described below, it was discovered that serum FFAs and their metabolites were highly increased in 37 lung adenocarcinoma patients compared to 111 matched non-cancer pulmonary patients. Areas under the receiver operating characteristic (ROC) curve range from 71%-82% (all P<0.001). In multivariable analysis, ROC increased to 87% (see Tables 1-3).

TABLE 1
Matched adenocarcinoma and control
patients: baseline characteristics

| | Cancer | Control | P | |
|---|---|---|---|---|
| Factor | (N = 37) | (N = 111) | value* | STD** |
| Male, n (%) | 24 (65) | 71 (64) | 0.92 | 0.02 |
| Smoking status, n (%) | | | 0.86F | 0.10 |
| Never | 1 (3) | 3 (3) | | |
| Former | 27 (73) | 76 (68) | | |
| Current | 9 (24) | 32 (29) | | |
| COPD, n (%) | 10 (27) | 32 (29) | 0.83 | −0.04 |
| DM, n (%) | 4 (11) | 13 (12) | 0.99F | −0.03 |
| Fam Hx Lung Ca, n (%) | 6 (16) | 21 (19) | 0.71 | −0.07 |
| Age, mean ± SD | 66.2 ± 11.4 | 65.4 ± 6.5 | 0.67 | 0.09 |
| Pack yrs, mean ± SD | 40 [25, 56] | 40 [26, 56] | 0.87 | −0.02 |
| Cancer stage | | | | |
| I | 7 (20) | — | | |
| II | 1 (3) | — | | |
| III | 12 (34) | — | | |
| IV | 15 (43) | — | | |

STD = standardized difference = difference in means or proportions/pooled standard deviation
*Pearson chi-square test, unless noted;
F = Fisher's exact test

TABLE 2
Adenocarcinoma - comparing cancer and controls for free fatty acids and phospholipids

| | | | | | Ratio of means~ | | P | |
|---|---|---|---|---|---|---|---|---|
| Factor | Cancer (N = 37) | | Control (N = 111) | | (99.7% CI) | | value \hat{ } | STD |
| HETE5 | 0.050 | [0.036, 0.1] | 0.0 31 | [0.0, 0.046] | 1.8 | (1.1, 3.2) | <0.001 * | 0.79 |
| HETE11 | 0.074 | [0.027, 0.114] | 0.032 | [0.019, 0.052] | 2.9 | (1.5, 5.5) | <0.001 * | 0.78 |
| HETE12 | 0.941 | [0.438, 1.729] | 0.234 | [0.126, 0.569] | 5.7 | (2.5, 13.0) | <0.001 * | 1.09 |
| HETE15 | 0.019 | [0.012, 0.024] | 0.009 | [0.005, 0.013] | 3.0 | (1.6, 5.4) | <0.001 * | 1.10 |
| AA | 5.700 | [4.64, 7.736] | 2.952 | [2.257, 4.629] | 2.3 | (1.6, 3.3) | <0.001 * | 1.34 |
| LA | 0.587 | [0.464, 0.726] | 0.334 | [0.225, 0.519] | 2.1 | (1.4, 3.2) | <0.001 * | 1.05 |
| HpODE_PC | 22.30 | [13.1, 33.3] | 17.87 | [12.3, 23.6] | TBD | | 0.029 | 0.41 |
| HODE_PC | 2.53 | [1.9, 5.5] | 7.06 | [3.2, 13.1] | | | <0.001 \hat{ } | −0.73 |
| AzPAF | 0.36 | [0.3, 0.5] | 0.46 | [0.3, 0.8] | | | 0.037 | −0.41 |
| C18lysoPAF | 12.66 | [8.6, 15.7] | 16.50 | [13.1, 19.8] | | | <0.001 \hat{ } | −0.68 |
| C16LysoPAF | 0.03 | [0.0, 0.0] | 0.04 | [0.0, 0.1] | | | 0.011 | −0.51 |

STD = standardized difference (difference in means or proportions/standard deviation)
\hat{ } 2-tailed Wilcoxon rank sum test
* Significant if P < 0.05/16 = 0.003
~estimated ratio of geometric means, calculated as exponentiated difference in means of the log- transformed data; 99.7% confidence interval maintains type I error at 5% across 8 variables.

TABLE 3
Adenocarcinoma: diagnostic accuracy of each FFA metabolite predicting lung cancer (N = 37 cancer, 111
controls)

| | | AUC | Best | | | | | |
|---|---|---|---|---|---|---|---|---|
| Variable | AUC (CI) | P-value | cutoff^{$} | PPV (CI)^{a} | NPV (CI)^{a} | Sensitivity(CI) | Specificity(CI) | Accuracy(CI) |
| AA | 0.82(0.71, | <0.001 | 4.63 | 0.24 (0.07, | 0.96 (0.9, 1.02) | 0.73 (0.51, | 0.74 (0.62, | 0.74 (0.63, |
| | 0.93) | | | 0.41) | | 0.95) | 0.87) | 0.85) |
| ia | 0.76 (0.64, | <0.001 | 0.48 | 0.23 (0.06, | 0.96 (0.9, 1.02) | 0.73 (0.51, | 0.73 (0.6, | 0.73 (0.62, |
| | 0.89) | | | 0.39) | | 0.95) | 0.85) | 0.84) |
| HETE12 | 0.78(0.65, | <0.001 | 0.51 | 0.2 (0.05, | 0.95 (0.89, | 0.69 (0.47, | 0.69 (0.56, | 0.69 (0.58, 0.8) |
| | 0.92) | | | 0.35) | 1.02) | 0.92) | 0.82) | |
| HETE15 | 0.78 (0.65, | <0.001 | 0.01 | 0.23 (0.06, | 0.96 (0.9, 1.02) | 0.73 (0.51, | 0.73 (0.6, | 0.73 (0.62, |
| | 0.92) | | | 0.39) | | 0.95) | 0.85) | 0.84) |
| HETE11 | 0.72 (0.56, | 0.001 | 0.04 | 0.19 (0.04, | 0.95 (0.88, | 0.68 (0.45, 0.9) | 0.67 (0.54, | 0.67 (0.56, |
| | 0.88) | | | 0.33) | 1.02) | | 0.8) | 0.79) |
| HETE5 | 0.71 (0.57, | <0.001 | 0.04 | 0.17 (0.03, | 0.94 (0.87, | 0.65 (0.42, | 0.65 (0.52, | 0.65 (0.54, |
| | 0.86) | | | 0.31) | 1.02) | 0.88) | 0.79) | 0.77) |
| HpODE_PC | 0.62 (0.45, | 0.040 | 19.86 | 0.16 (0.02, | 0.94 (0.86, | 0.62 (0.39, | 0.62 (0.49, | 0.62 (0.5, 0.74) |
| | 0.79) | | | 0.29) | 1.02) | 0.86) | 0.76) | |
| HODE_PC | 0.7 (0.55, | <0.001 | 4.19 | 0.17 (0.03, | 0.94 (0.87, | 0.65 (0.42, | 0.65 (0.52, | 0.65 (0.53, |
| | 0.85) | | | 0.31) | 1.02) | 0.88) | 0.79) | 0.77) |
| AzPAF | 0.62 (0.46, | 0.027 | 0.41 | 0.13 (0.01, | 0.92 (0.83, | 0.57 (0.33, | 0.57 (0.43, | 0.57 (0.45, |
| | 0.77) | | | 0.25) | 1.01) | 0.81) | 0.71) | 0.69) |
| C18lyso | 0.69 (0.53, | <0.001 | 14.72 | 0.18 (0.03, | 0.94 (0.87, | 0.65 (0.42, | 0.66 (0.53, | 0.66 (0.54, |
| PAF | 0.85) | | | 0.32) | 1.02) | 0.88) | 0.79) | 0.77) |

FFA metabolite values are metabolite peak/internal standard peak
PPV = positive predictive value;
NPV = negative predictive value
^{a}estimate using Bayes¹ Theorem assuming a true prevalence of 0.10.
AUC = area under the receiver operating characteristic curve.
AUC P-value: testing H0:
AUC = 0.5 (1-tailed).
^{$}Jointly maximizes sensitivity and specificity. Diagnostic accuracy parameters (sensitivity, specificity, etc.) correspond to the given threshold value.
CI estimated using bootstrap resampling (percentile method) with 5,000 resamples. Accuracy; ratio of sum of true positives, true negatives, false positives, false negatives to total sample size. Data values represent peak raw biomarker values divided by the corresponding internal standard for each sample, N = 3 biomarkers met the criteria of having sensitivity and specificity at the best outpoint > 0.70 (AA, LA, HETE15).
CI: 99.64% confidence intervals adjusted for assessing multiple biomarkers using Bonferroni correction; overall alpha = 0.05

### EXAMPLE 2

### Methods

Forty (40) prostate cancer patients with mean±SD age of 64±6 years old were compared to eighty-seven (87) male lung clinic patients (67±6 years old) on ten (10) biomarkers (FFA metabolites). Blood samples were collected before patients had potential curative surgeries. Seven (7) patients had neoadjuvant chemotherapy with a taxane before surgery. At the time of sampling, patients were at early stages (Surgical Gleason Score at 6 or 7). Mass spectrometric analyses on the ten (10) biomarkers were performed as described above in Example 1.

### Results

Table 4 gives the area under the receiver operating characteristic curve (AUC) and 95% Cl for each FFA metabolite.

TABLE 4
1. Prostate Cancer vs. Non-Cancer Controls

| | Prostate | | Non-Cancer | | | | | | |
|---|---|---|---|---|---|---|---|---|---|
| | CA | | Controls | | | | | | |
| | (N = 40) | | (N = 87) | | Odds | | | | P value |
| | Median | | Median | | ratio* | | AUC\hat{ } | | (2-sided) |
| Factor** | [q1, q3] | | [q1, q3] | | (95% CI) | | (95% CI) | | H0: AUC = 0.5 |
| AA_S | 3.98 | [3.33, 6.06] | 2.99 | [2.26, 4.57] | 2.6 | (1.4, 4.7) | 0.71 | (0.62, 0.80) | 0.001 |
| LA_S | 0.58 | [0.51, 0.73] | 0.32 | [0.22, 0.53] | 4.7 | (2.4, 9.3) | 0.81 | (0.73, 0.88) | <0.001 |
| HETE5_S | 0.08 | [0.06, 0.17] | 0.03 | [0.02, 0.05] | 5.5 | (2.9, 10.6) | 0.88 | (0.82, 0.94) | <0.001 |
| HODE9_S | 0.86 | [0.47, 1.20] | 0.57 | [0.29, 0.86] | 1.8 | (1.2, 2.6) | 0.68 | (0.58, 0.78) | <0.001 |
| HETE11_S | 0.07 | [0.04, 0.13] | 0.03 | [0.02, 0.05] | 2.5 | (1.7, 3.7) | 0.78 | (0.69, 0.87) | <0.001 |
| HETE12_s | 0.11 | [0.05, 0.19] | 0.21 | [0.11, 0.59] | 0.6 | (0.5, 0.9) | 0.31 | (0.21, 0.41) | <0.001 |
| HODE13_S | 0.74 | [0.43, 1.22] | 0.45 | [0.30, 0.69] | 1.8 | (1.3, 2.5) | 0.68 | (0.58, 0.79) | <0.001 |
| HETE15_S | 0.02 | [0.01, 0.05] | 0.01 | [0.01, 0.01] | 3.7 | (2.3, 6.4) | 0.84 | (0.78, 0.91) | <0.001 |

*odds ratio of cancer for doubling in predictor;
**all predictors standardized
\hat{ }AUC (area under the receiver operating characteristic curve) estimates the predictive ability of biomarker, and typically ranges from 0.50 (chance) to 1 (perfect discrimination); values below 0.50 indicate lower values, on average, for cancer patients than controls
AA (arachidonate); LA (linoleate)

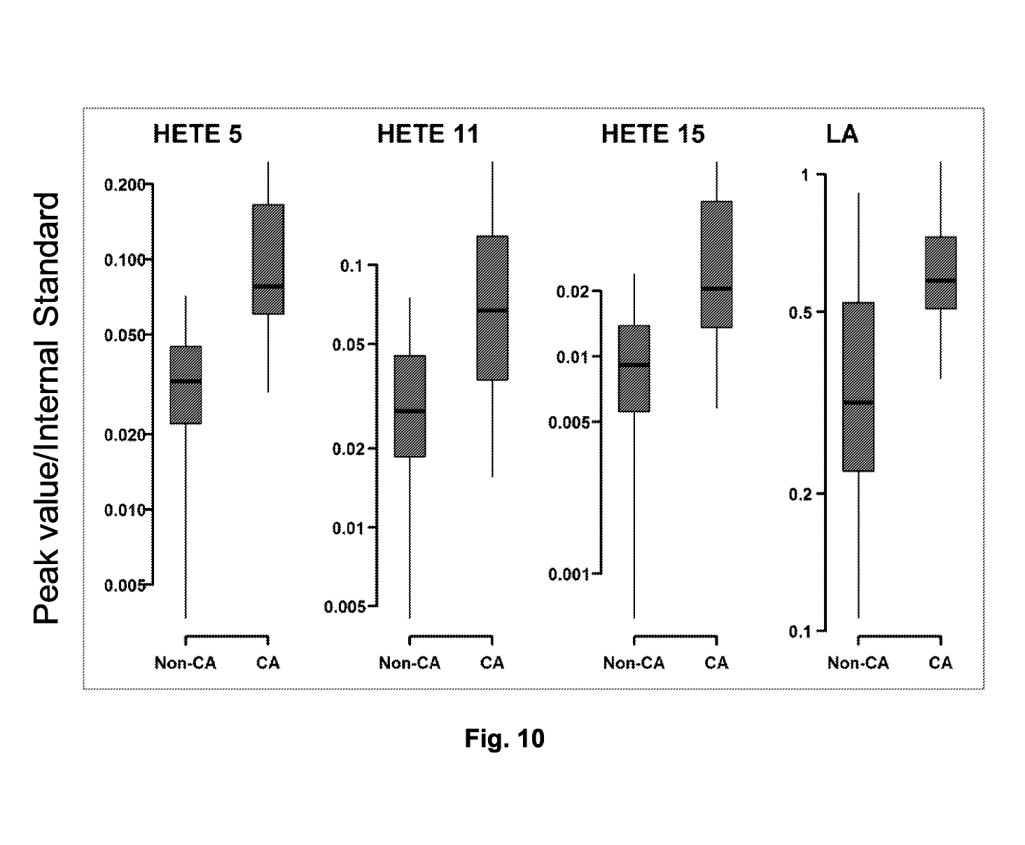

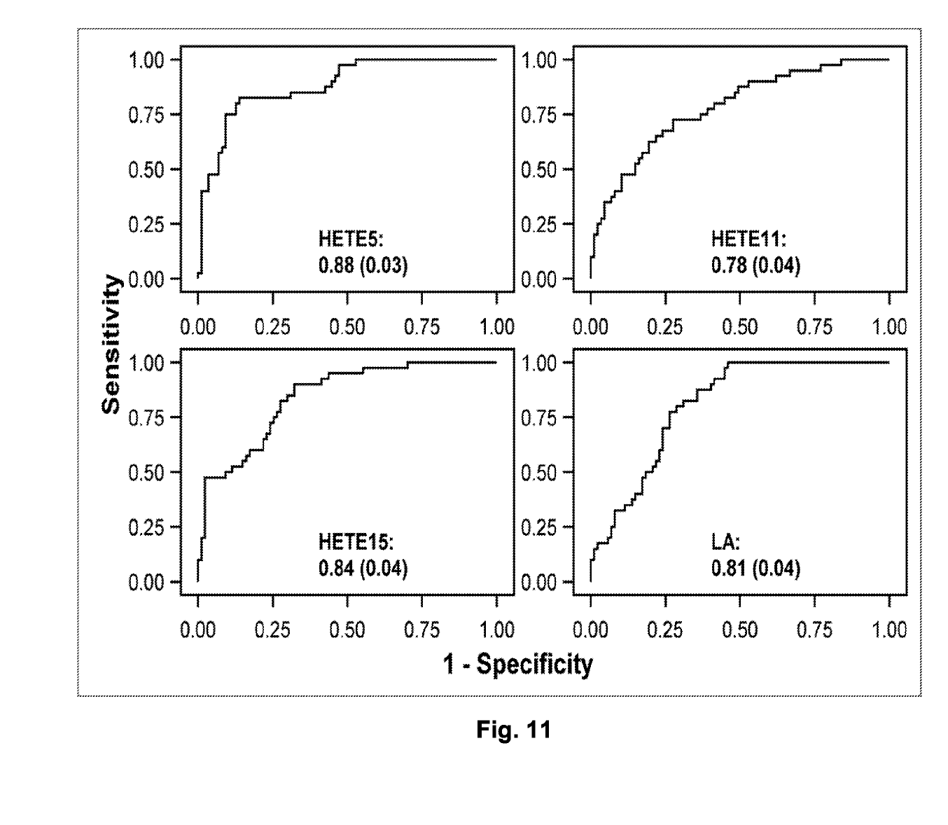

All biomarkers were significantly different between the groups (FIGS. 10-11), with AUC ranging from M8 to 0.88 and another (HETE12) significantly below 0.5. Estimated odds ratios of having cancer for a doubling in the biomarker ranged from 1.8 to 5.5 for 7 biomarkers with odds ratios above 1.0, and 0.66 for HETE12, all statistically significant (i.e., different from 1.0) at the 0.05 significance level.

### EXAMPLE 3

### Methods

With IRB approval and informed consent, we enrolled 15 patients with colon cancer who were scheduled for potentially curative tumor resection. We also enrolled 20 patients without known cancer who were scheduled for spine surgery.

Demographic and morphometric characteristics were recorded, as were anesthetic and surgical details. Venous blood was sampled before surgery, and again 24-hours postoperatively. Samples were centrifuged at 3000 times G, and the resulting serum was frozen at −70° C. until assayed. Mass spectrometric analyses on the ten (10) biomarkers were performed as described above in Example 1.

### Results

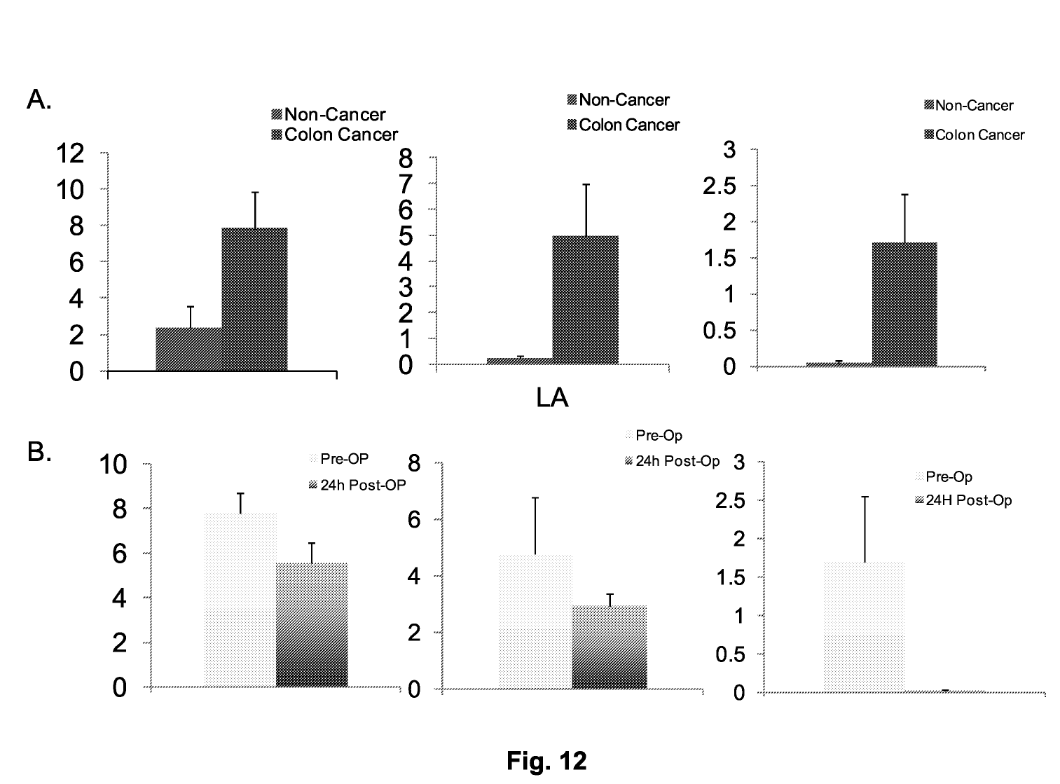

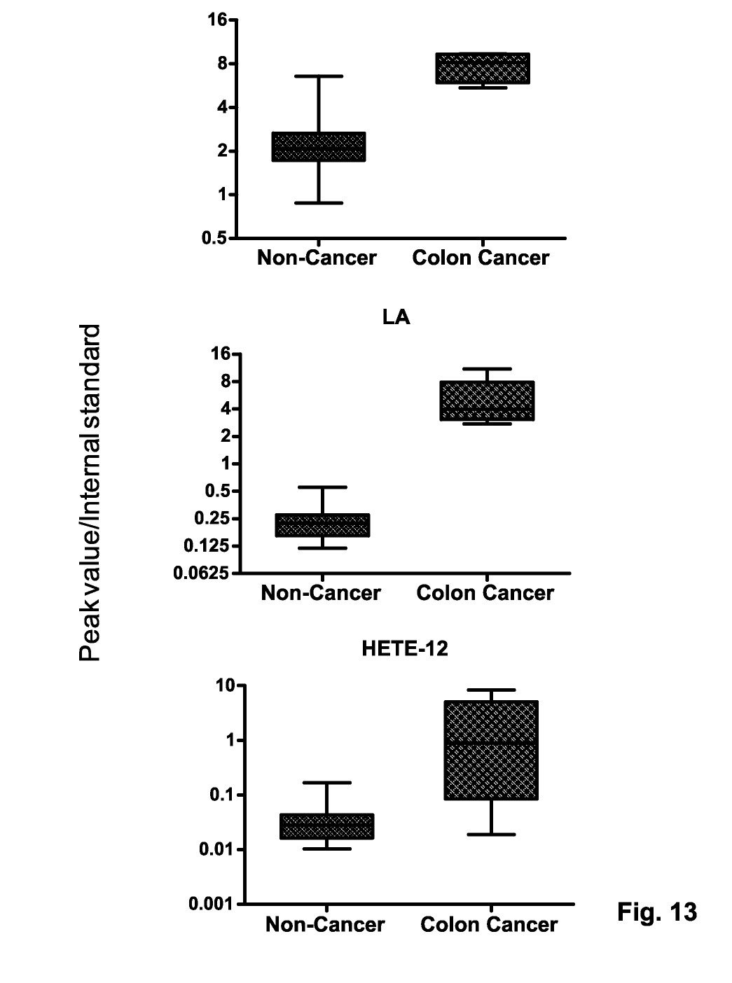

As shown in FIGS. 12A-B and FIG. 13, means of AA, LA, and HETE-12 were increased 4, 20, and 40 times in colon cancer compared to non-cancer controls (respectively). Statistics were very significant, all p<0.001. Twenty-four hours after surgery, these biomarkers were significantly decreased compared to pre-surgery.

### EXAMPLE 4

### Methods

Thirty-one (31) renal cancer patients whose blood samples were collected before patients had potential curative surgeries were collected. Twenty non-cancer patient blood samples were also collected before spine surgery. Mass spectrometric analyses on the ten (10) biomarkers were performed as described above in Example 1.

### Results

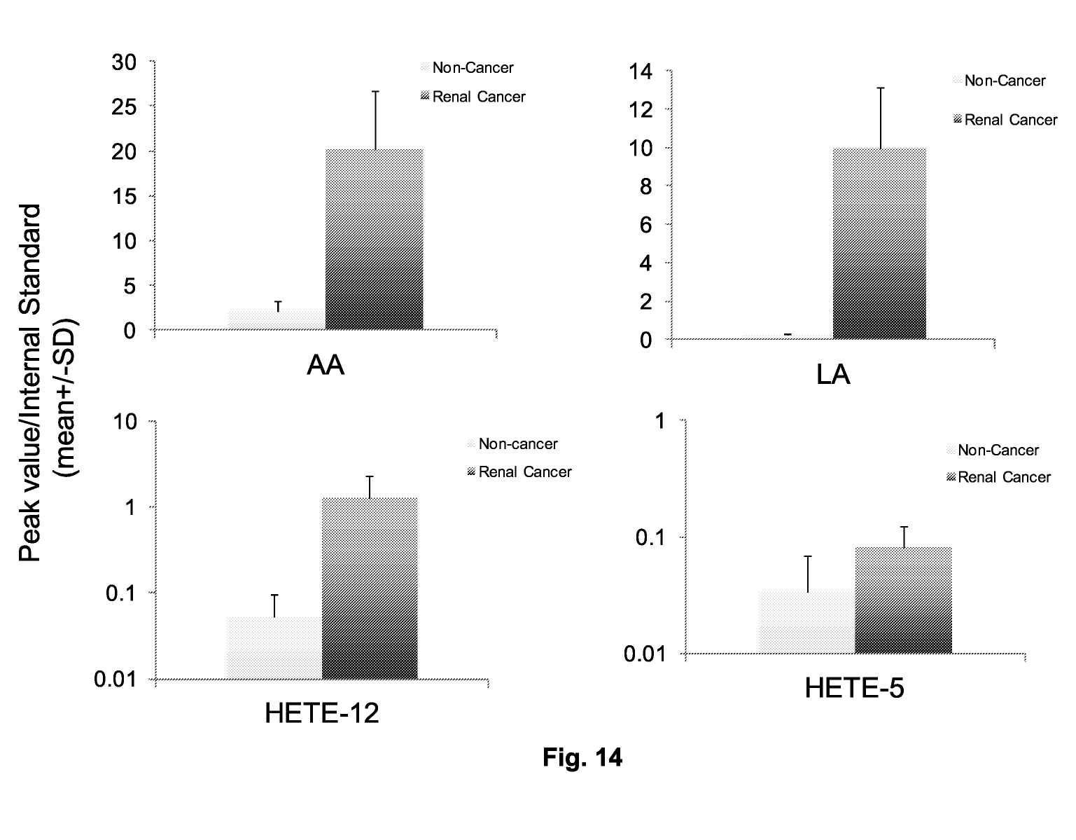

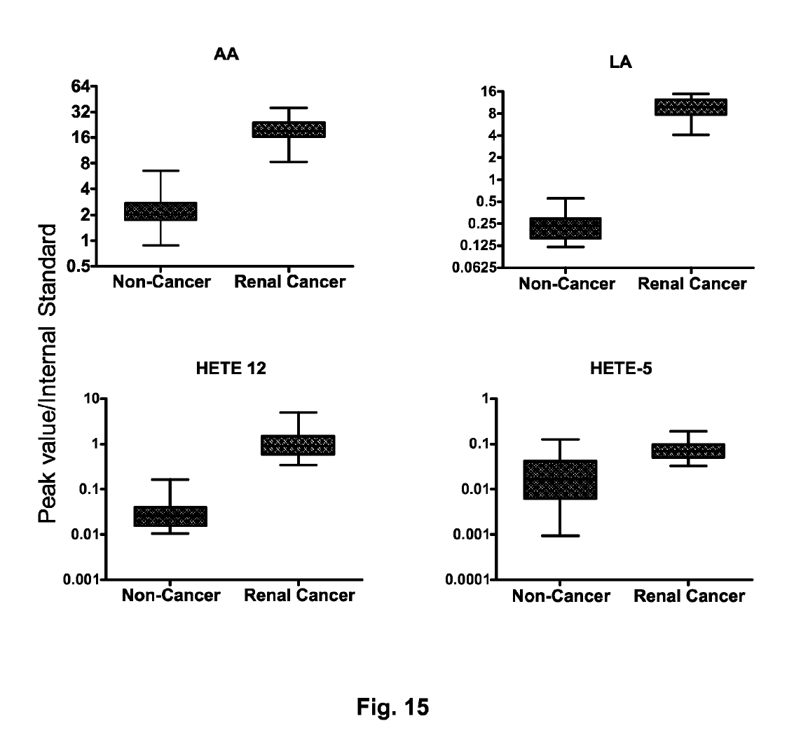

As shown in FIGS. 14-15, means of AA, LA, HETE-12, and HETE-5 were increased 10, 50, 31, and 3 times (respectively) in renal cancer compared to non-cancer controls.

From the above description of the present disclosure, those skilled in the art will perceive improvements, changes and modifications. Such improvements, changes, and modifications are within the skill of one in the art and are intended to be covered by the appended claims.

The following is claimed:

1. A method for detecting cancer in a subject from a biological sample previously withdrawn from the subject, the method comprising determining in vitro the level of at least one biomarker in the biological sample, the at least one biomarker including a phospholipid or a free fatty acid, wherein a level of the at least one biomarker that is at least about 2-fold greater than the level of at least one biomarker in a control is indicative of cancer in the subject.

2. The method of claim 1, wherein the biological sample is selected from the group consisting of whole blood, plasma and serum.

3. The method of claim 2, wherein the biological sample is about 0.5 ml of blood.

4. The method of claim 1, wherein the level of the at least one biomarker in the subject is determined using mass spectroscopy.

5. The method of claim 4, wherein the level of the at least one biomarker in the subject is determined using electrospray ionization tandem mass spectroscopy.

6. The method of claim 1, wherein the free fatty acid is at least one of linoleic acid (LA) or arachidonic acid (AA).

7. The method of claim 1, wherein the free fatty acid is at least one of a hydroxyeicosatetraenoic acid (HETE) or a hydroxyoctadecadienoic acid (HODS).

8. The method of claim 7, wherein the HETE is selected from the group consisting of 15-HETE, 12-HETE, 11-HETE and 5-HETE.

9. The method of claim 1, wherein the phospholipid is a lysophosphatylcholine (LPC) or HODE-PC.

10. The method of claim 9, wherein the LPC is LPC-C16.

11. The method of claim 7, wherein the level of HETE is at least about 8-fold higher than the control level.

12. The method of claim 7 wherein the level of HODE is at least about 12-fold higher than the control level.

13. The method of claim 9, wherein the level of LPC is at least about 2-fold higher than the control level.

14. The method of claim 9, wherein the level of HODE-PC is at least about 2-fold lower than the control level.

15. The method of claim 1, wherein the cancer is early stage cancer.

16. The method of claim 15, wherein the early stage cancer is one of lung cancer, prostate cancer, colon cancer or renal cancer.

17. A method for predicting the prognosis of a subject with cancer following a medical intervention to treat the cancer from a biological sample previously withdrawn from the subject, said method comprising determining in vitro the level of at least one biomarker in the biological sample, the at least one biomarker including a phospholipid or a free fatty acid, wherein a decreased level of the at least one biomarker as compared to a pre-treatment level of at least one biomarker is indicative of a favorable prognosis.

18. The method of claim 17, wherein the medical intervention substantially eliminates the cancer.

19. The method of claim 17, wherein the medical intervention includes surgically resecting at least one tumor in the subject.

20. The method of claim 19, wherein said determining step is performed about 6 hours after said resecting step.

21. The method of claim 20, wherein the level of the at least one biomarker is decreased at least about 2-fold compared to the pre-treatment level.

22. The method of claim 19, wherein said determining step is performed about 24 hours after said resecting step.

23. The method of claim 22, wherein the level of the at least one biomarker is decreased at least about 3-fold compared to the pre-treatment level.

24. A method for detecting cancer recurrence in a subject following a medical intervention to treat the cancer from a biological sample previously withdrawn from the subject, said method comprising determining in vitro the level of at least one biomarker in the biological sample over a period of time, the at least one biomarker including a phospholipid or a free fatty acid, wherein a level of the at least one biomarker that is at least about 2-fold higher than a pre-treatment level of at least one biomarker over the period of time is indicative of cancer recurrence in the subject.

25. The method of claim 24, wherein the medical intervention includes resection of at least one tumor from the subject.
